(19) United States
(12) Patent Application Publication
(10) Pub. No.: US 2025/0144449 A1
(43) Pub. Date: May 8, 2025

(54) STIMULATION OF THE CELIAC-SUPERIOR MESENTERIC GANGLION COMPLEX FOR TREATING INFLAMMATION

(71) Applicant: The Feinstein Institutes for Medical Research, Manhasset, NY (US)

(72) Inventors: Valentin A. Pavlov, Manhasset, NY (US); Santhoshi Poonacha Palandira, Manhasset, NY (US); Kevin J. Tracey, Manhasset, NY (US)

(73) Assignee: The Feinstein Institutes for Medical Research, Manhasset, NY (US)

(21) Appl. No.: 18/929,926

(22) Filed: Oct. 29, 2024

Related U.S. Application Data
(60) Provisional application No. 63/595,832, filed on Nov. 3, 2023.

(51) Int. Cl. A61N 7/02; A61N 7/00

(52) U.S. Cl. CPC A61N 7/02; A61N 2007/0026

(57) ABSTRACT

Methods are disclosed for treating inflammatory disease states in a mammalian patient (e.g., human) by applying noninvasive focused ultrasound stimulation to the celiac-superior mesenteric ganglion complex of the patient.

### CROSS-REFERENCE TO RELATED APPLICATION

This application claims the benefit of U.S. Provisional Patent Application No. 63/595,832, filed on Nov. 3, 2023, the contents of which are herein incorporated by reference in their entirety into the present application.

### STATEMENT OF GOVERNMENT SUPPORT

This invention was made with government support under grant number GM128008 awarded by the National Institutes of Health. The government has certain rights in the invention.

### FIELD OF THE INVENTION

The present invention is based on applying noninvasive focused ultrasound stimulation (FUS) to the celiac-superior mesenteric ganglion complex (CSMGC) for non-invasive treatment of inflammatory disease states.

### INCORPORATION BY REFERENCE

All publications and patent applications mentioned in this specification and all publications cited in said publications and patent applications are herein incorporated by reference in their entirety to the same extent as if each individual publication or patent application was specifically and individually indicated to be incorporated by reference.

### BACKGROUND OF THE INVENTION

Inflammation is a vital protective event against tissue injury and pathogen invasion, which helps to maintain homeostasis. Cytokines, including tumor necrosis factor (TNF), are important molecular mediators of inflammation. However, dysregulated, and excessive acute and chronic inflammation and increased TNF and other pro-inflammatory cytokine levels are implicated in the pathophysiology of various diseases and conditions, including sepsis, rheumatoid arthritis, Crohn's disease, cancer, ankylosing spondylitis, atherosclerosis, cardiac and cerebral ischemia, vasculitis, Alzheimer's disease, multiple sclerosis, systemic lupus erythematosus, obesity, diabetes, and asthma. Hence, it is important to control inflammation and suppress various forms of aberrant inflammation.

The inflammatory reflex is a main physiological vagus nerve-mediated mechanism that regulates inflammation^{1, 2}. Recently, the celiac-superior mesenteric ganglion complex (CSMGC) has been identified as an important relay center in the efferent arm of the vagus nerve-based immunoregulatory mechanisms of the inflammatory reflex. The efferent arm of the inflammatory reflex—the cholinergic anti-inflammatory pathway is based on the efferent vagus nerve, which originates in the brainstem dorsal motor nucleus of the vagus (DMN) and nucleus ambiguus (NA) and interacts in the (CSMGC) in the abdomen with the cellular bodies of catecholaminergic neurons innervating the spleen, liver and other organs³⁻⁶.

Sympathetic preganglionic fibers also innervate the CSMGC and interact with catecholaminergic neurons. The CSMGC is located ventral to the aorta, medial to the upper pole of the left kidney. Studies have demonstrated the anti-inflammatory efficacy of vagus nerve stimulation (VNS) in animals and humans with inflammatory conditions⁷⁻⁹. However, no previous studies have investigated the effects of direct neuromodulation of the CSMGC on cytokine levels and inflammation and in treating inflammatory disorders.

Focused ultrasound stimulation (FUS) is a noninvasive technique of neuromodulation which allows for precise and localized stimulation or inhibition of neurons, based on the parameters of energy applied. FUS can be applied to both central and peripheral neuronal structures.

The present invention reveals that noninvasive stimulation of the CSMGC is an efficient novel approach to suppress aberrant inflammation. This approach indicates new possibilities to control inflammation by modulating CSMGC neuronal populations. These and other objectives are achieved by the present invention discussed below.

### SUMMARY OF THE INVENTION

The invention provides a non-invasive method to treat an inflammatory disease state in a mammalian patient comprising applying noninvasive focused ultrasound stimulation (FUS) to the patient's celiac-superior mesenteric ganglion complex (CSMGC) in an amount effective to treat the inflammatory disease state in a mammalian patient.

In different embodiments, the inflammatory disease state is, for example, but not limited to, one or more of sepsis, endotoxemia, septicemia, septic shock, rheumatoid arthritis, Crohn's disease, cancer, ankylosing spondylitis, cardiac ischemia, cerebral ischemia, vasculitis, Alzheimer's disease, multiple sclerosis, systemic lupus erythematosus, obesity, asthma, diabetes, acute liver injury, non-alcoholic steatohepatitis, and liver disease.

The invention also provides a method of one or both of suppressing tumor necrosis factor (TNF) levels in a mammalian patient and increasing interleukin-10 (IL-10) levels in the mammalian patient comprising applying noninvasive focused ultrasound stimulation (FUS) to the patient's celiac-superior mesenteric ganglion complex (CSMGC) in an amount effective to suppress TNF levels and/or increase IL-10 levels in a mammalian patient.

The invention further provides a method of enhancing an inflammatory response in a mammalian patient comprising applying noninvasive focused ultrasound stimulation (FUS) to the patient's celiac-superior mesenteric ganglion complex (CSMGC) in an amount effective to inhibit or ablate neurons and nerve endings in the CSMGC of a mammalian patient.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. FUS of the CSMGC of mice attenuates serum TNF levels in murine endotoxemia. LPS, lipopolysaccharide; TNF, tumor necrosis factor. C57BL6/J male mice were subjected to sham stimulation or FUS (5 mins) of the CSMGC. Animals were allowed to recover for 5 mins and LPS (0.25 mg/kg, i.p.) was administered. Blood was obtained 90 min post-LPS administration and serum TNF levels were analyzed using ELISA. Data are represented as individual mouse data points with mean ±SEM. Unpaired t test: sham stimulation vs FUS stimulation.

FIG. 2. FUS of the CSMGC of mice attenuates hepatic TNF levels in murine endotoxemia. C57BL6/J male mice were subjected to sham stimulation or FUS (5 mins) of the CSMGC. Animals were allowed to recover for 5 mins and LPS (0.25 mg/kg, i.p.) was administered. Blood was obtained 90 min post-LPS administration and liver (hepatic) TNF levels were analyzed using ELISA. Data are represented as individual mouse data points with mean ±SEM. Unpaired t test: sham stimulation vs FUS stimulation.

FIG. 3. Noninvasive focused ultrasound stimulation (FUS) of CSMGC significantly increases serum IL-10 levels during endotoxemia. C57BL6/J male mice were subjected to sham stimulation or FUS (5 mins) of the CSMGC. Animals were allowed to recover for 5 mins and LPS (0.25 mg/kg, i.p.) was administered. Blood was obtained 90 min post-LPS administration and serum IL-10 levels were analyzed using milliplex assay. Data are represented as individual mouse data points with mean ±SEM. Unpaired t test: sham stimulation vs FUS stimulation.

FIGS. 4A-4B. FUS of the CSMGC area in male mice subjected to ganglionic complex surgical removal (ganglionectomy) does not alter (A) serum and (B) hepatic TNF levels during endotoxemia. Male ChAT-tdTomato mice were subjected to celiac-superior mesenteric ganglionectomy. Animals were allowed to recover for 1 week and then underwent FUS (5 mins) of the CSMGC area. After 5 mins they received LPS (0.25 mg/kg, i.p.). Serum and liver were obtained 90 min post-LPS administration and TNF levels were analyzed using ELISA. Data are represented as individual mouse data points with mean ±SEM. Unpaired t test: sham stimulation vs FUS.

FIGS. 5A-5B. FUS of the CSMGC area in female mice subjected to ganglionic complex surgical removal (ganglionectomy) does not alter (A) serum and (B) hepatic TNF levels during endotoxemia. Female ChAT-tdTomato mice were subjected to celiac-superior mesenteric ganglionectomy. Animals were allowed to recover for 1 week and then underwent FUS (5 mins) of the CSMGC area. After 5 mins they received LPS (0.25 mg/kg, i.p.). Serum and liver were obtained 90 min post-LPS administration and TNF levels were analyzed using ELISA. Data are represented as individual mouse data points with mean ±SEM. Unpaired t test: sham stimulation vs FUS.

### DETAILED DESCRIPTION

The invention provides non-invasive methods to treat an inflammatory disease state in a mammalian patient comprising applying noninvasive focused ultrasound stimulation (FUS) to the patient's celiac-superior mesenteric ganglion complex (CSMGC) in an amount effective to treat the inflammatory disease state in a mammalian patient (e.g. human or non-human animals, such as companion animals (e.g., cats and dogs), equines, porcines, etc.).

The CSMGC is located ventral to the aorta, medial to the upper pole of the left kidney. The efferent vagus nerve innervates the CSMGC, and these innervations are cholinergic. The CSMGC is also innervated by sympathetic preganglionic cholinergic neurons, including the splanchnic nerves. From the CSMGC there are postganglionic fibers that relay to the spleen, liver and other organs such as kidney, stomach, intestines, and pancreas. These anatomical considerations make the CSMGC a very attractive, but previously unexplored, site to apply neuromodulation to control inflammation. Ultrasonically modulating the CSMGC is done noninvasively by placing a focused ultrasound probe on the surface of the abdomen in the area over and/or in proximity to the CSMGC of the mammalian patient or subject.

In different embodiments, the inflammatory disease state is, for example, but not limited to, one or more of sepsis, endotoxemia, septicemia, septic shock, rheumatoid arthritis, Crohn's disease, cancer, ankylosing spondylitis, cardiac ischemia, cerebral ischemia, vasculitis, Alzheimer's disease, multiple sclerosis, systemic lupus erythematosus, obesity, asthma, diabetes, acute liver injury, non-alcoholic steatohepatitis, and liver disease. In one embodiment, the inflammatory disease state is systemic inflammatory or inflammation of the liver.

Treating an inflammatory disease state means to ameliorate one or more signs or symptoms of the inflammatory disease state.

In different embodiments, application of FUS to the patient's CSMGC suppresses tumor necrosis factor (TNF) levels and/or elevates interleukin-10 (IL-10) levels in the patient. In different embodiments, application of FUS to the patient's CSMGC does one or more of the following: suppress tumor necrosis factor (TNF) levels in the patient's serum, suppress TNF levels in the patient's liver, and elevate interleukin-10 (IL-10) levels in the patient's serum.

The inventors hypothesized that activating structures within the CSMGC might be sufficient to activate anti-inflammatory signaling. As noted above, efferent signals originating in the DMN are carried by cholinergic fibers in the vagus nerve and interact in the CSMGC with the adrenergic cell bodies of the splenic nerve. The norepinephrine released by the splenic nerve acts on the choline acetyltransferase (ChAT) positive T cells in the spleen to release acetylcholine. In turn, the released acetylcholine interacts with the α7nAChR receptors to suppress the release of proinflammatory cytokines. In addition, the sympathetic preganglionic cholinergic fibers innervating the CSMGC also play a role in the regulation of inflammation. As the CSMGC is an important center of regulatory output to the liver and other abdominal organs, the inventors expected that applying FUS to the CSMGC would affect the TNF and other cytokine levels in the organs associated with the CSMGC.

The invention also provides methods of one or both of suppressing tumor necrosis factor (TNF) levels in a mammalian patient and increasing interleukin-10 (IL-10) levels in the mammalian patient comprising applying noninvasive focused ultrasound stimulation (FUS) to the patient's celiac-superior mesenteric ganglion complex (CSMGC) in an amount effective to suppress TNF levels and/or increase IL-10 levels in a mammalian patient. In one embodiment, TNF levels are suppressed in the mammalian patient's liver and/or serum. In one embodiment, IL-10 levels are increased in the mammalian patient's serum. In one embodiment, the invention provides a method of reducing tumor necrosis factor (TNF) release by macrophages/Kupffer cells in a patient by applying FUS ultrasound to cholinergic neuronal endings and adrenergic (catecholaminergic) cell bodies in the CSMGC of a mammalian patient (e.g., human) in an amount effective to reduce TNF release by macrophages/Kupffer cells.

In different embodiments, the mammalian patient has an inflammatory disease state. For example, where the inflammatory disease state is one or more of sepsis, endotoxemia, septicemia, septic shock, rheumatoid arthritis, Crohn's disease, cancer, ankylosing spondylitis, cardiac ischemia, cerebral ischemia, vasculitis, Alzheimer's disease, multiple sclerosis, systemic lupus erythematosus, obesity, diabetes, asthma, acute liver injury, non-alcoholic steatohepatitis, and liver disease.

The invention further provides methods of enhancing an inflammatory response in a mammalian patient comprising applying noninvasive focused ultrasound stimulation (FUS) to the patient's celiac-superior mesenteric ganglion complex (CSMGC) in an amount effective to inhibit or ablate neurons and nerve endings in the CSMGC of a mammalian patient. In different embodiments, application of FUS to the mammalian patient's CSMGC increases serum levels of tumor necrosis factor (TNF) in the mammalian patient and/or decreases serum levels of interleukin-10 (IL-10) in the mammalian patient.

In preferred embodiments, the mammalian patient is a human.

In alternative embodiments, the mammalian patient is a companion animal.

We have specified the parameters of FUS we have used in our studies to reliably attenuate serum and hepatic TNF and increase serum IL-10 during murine endotoxemia in the Experimental Details and Results. Of note, studies using FUS applied to other parts of the body have established a range of parameters and regimens that can be reliably applied to the CSMGC to achieve neuronal activation or neuronal or general tissue ablation. In both strategies ultrasound guidance can be used. As previously summarized neuronal activation to inhibit inflammation can be achieved using acute and chronic paradigms. For instance FUS using 1 MHz US at 350 kPa, 1 s on/5 s off bursts for 2 min per day has anti-inflammatory effects in mice¹⁰. Splenic FUS using sine waves with a frequency of 1.1 MHZ, amplitude of 200 mVpp, burst cycles of 150, and a burst period of 200 ms results in anti-inflammatory effects¹¹. In humans, applying FUS in a range of energy intensities (i.e., 11.7-225.4 mW/cm²) can inhibit TNF levels¹². As previously characterized, these FUS parameters create energy, which is significantly lower than the one associated with ablation (35 W/cm² for ablation)¹³. High intensity focused ultrasound can cause tissue ablation and has been utilized for CNS/brain and peripheral targets. Non-invasive high intensity FUS ablation has been also exploited in the treatment of several tumors cancers including breast cancer^{14, 15, 16} and various parameters have been used. Ultrasound guided high intensity FUS was also implemented for treating uterine fibroids¹⁷. High intensity FUS treatments were performed in continuous wave a mean acoustic power of 274.6±53.5 W (range: 185-369 W); a mean exposure time of 29.5±11.9 minutes (range: 11.4-46.6 minutes), and a mean acoustic energy was 444.7±182.7 KJ (range: 184-734 KJ)¹⁷. The mean treatment duration was 113.3±41.2 mins.¹⁴⁻¹⁶ Using non-invasive ultrasound guided high intensity FUS can be successfully used for CSMGC ablation in therapeutic inflammatory settings for increasing TNF levels.

Ultrasound guidance can be utilized in both strategies, i.e., non-invasive FUS stimulation of the CSMGC and high intensity FUS for CSMGC ablation. For instance, we have performed FUS stimulation of the CSMGC as follows: Mice were anesthetized and placed in supine position, and the abdomen was surgically prepped. The ultrasound probe was placed over the abdomen and the medial aspect of the left kidney was identified. Using the ultrasound doppler mode, on the medial aspect of the left kidney, the confluence of the aorta, IVC and the left renal vein was identified. This was the landmark for the CSMGC, which was marked. Then the FUS transducer was placed over this area and stimulation was delivered.

All combinations of the various elements described herein are within the scope of the invention unless otherwise indicated herein or otherwise clearly contradicted by context.

This invention will be better understood from the Experimental Details, which follow.

### Experimental Details

Focused ultrasound stimulation (FUS) is a noninvasive technique of neuromodulation that allows for precise and localized stimulation or inhibition of neurons, based on the parameters of energy applied^{7,18}. FUS can be applied to both central and peripheral neuronal structures^{7, 19, 20}. Here we used FUS to stimulate the neuronal endings and the neuronal bodies within the CSMGC.

The inventors hypothesized activation of the structures within the CSMGC is sufficient to activate anti-inflammatory signaling within the inflammatory reflex. It is known that efferent signals originating in the dorsal motor nucleus of the vagus (DMN) are carried by cholinergic fibers in the vagus nerve and synapse in the CSMGC with the adrenergic cell bodies of the splenic nerve. The norepinephrine released by the splenic nerve act on the choline acetyltransferase (ChAT) positive T cells in the spleen to release acetylcholine, which in turn acts on the α7nAChR to suppress the release of proinflammatory cytokines (5,6). The signals which originate on stimulating the cholinergic neuronal endings and the catecholaminergic neuronal bodies in the CSMGC are sufficient to regulate systemic inflammation in response to endotoxemia in a mouse model.

### Methods

Animals: C57BL6/J mice were purchased from Jackson Laboratories (C57BL6/J, Stock#000664). ChAT-tdTomato mice were bred in house after crossing a ChAT-Cre (stock#006410) male mice with a ROSA-tdTomato (stock#007914) female mice, both purchased from Jackson laboratories. Animals were kept at 25° C. on a 12-hour light/dark cycle. Water and regular rodent chow were available ad libitum. All experiments were performed under protocols approved by the Institutional Animal Care and Use Committee of the Feinstein Institute for Medical Research, Northwell Health.

CSMGC FUS stimulation: Mice were anesthetized using isoflurane. After achieving appropriate depth of anesthesia, the abdomen was shaved and hair removal cream was applied, following which the area was prepped with alcohol and betadine wipes. The mouse was placed supine and using the ultrasound imaging probe, the left kidney was identified and medial to it the confluence of the left renal vein and the branch of the aorta was identified, which is the location of the CSMGC based on the landmarks. Then the focused ultrasound transducer probe (Sonic Concepts) was placed at that site and FUS stimulation was delivered for a total of 5 mins period of stimulation. It was delivered as 1 min (1.1 MHZ, 200 mV per pulse, 150 burst cycles, 500 μs burst period) of stimulation followed by 30 sec period of rest. At the end of stimulation, mice were placed on a heating pad until complete recovery.

Celiac-superior mesenteric ganglionectomy: Mice were anesthetized using isoflurane. After achieving appropriate depth of anesthesia, the abdomen was shaved and hair removal cream was applied, following which the area was prepped with alcohol and betadine wipes. The mouse was placed supine, and a midline vertical incision placed over the abdomen. The intestines were moved aside to visualize the left kidney, aorta, and its branch—superior mesenteric artery and left renal vein. The CSMGC is visible as bright red structure under the fluorescent stereomicroscope at this landmark. The left splanchnic nerve was sectioned and then the CSMGC was held with forceps and released from the surrounding connective tissues. The right splanchnic nerve was sectioned from the right celiac ganglia and the entire CSMGC was removed. Of note, there was no damage to the vascular structures in the vicinity. The field was examined for any complications under the bright field microscope and then the abdomen was sutured in layers. Mice were given analgesic for pain relief and a single dose of saline subcutaneously. They were allowed to recover under the heat lamp completely.

Endotoxemia: A stock solution of lipopolysaccharide (LPS) from Escherichia coli 0111:B4 (Sigma-Aldrich #L5293) was kept −20° C. at a concentration of 1 mg/ml. Prior to use, the stock solution was thawed, diluted and sonicated to prevent adhesion of the LPS molecules. LPS was administered via intraperitoneal injection to each mouse at a dose of 0.25 mg/kg body weight. Ninety minutes following injection, they were euthanized, and blood, spleen, kidneys, pancreas, and liver were collected. Blood was allowed to clot at room temperature for 45 minutes. Blood was then centrifuged at 5,000 rpm for 10 minutes and the supernatant (debris free serum) serum was analyzed for tumor necrosis factor (TNF) using commercially available ELISA (Invitrogen #88-7324-22) and other cytokines, chemokines and analytes were assayed using Milliplex Mouse cytokine/chemokine magnetic bead panel—Immunology multiplex assay. The liver was homogenized using zirconium beads in a homogenizer and then centrifuged at 5,000 Rpm for 5 minutes, supernatant was collected, which was then centrifuged at 10,000 rpm for 2 minutes. The resulting supernatant fluid was collected and analyzed for TNF using the above-mentioned ELISA kit and for proteins using Bradford protein assay.

### Results

To investigate the hypothesis that stimulation of the neuronal endings and cell bodies within the CSMGC attenuates inflammation, we used commercially available C57BL6/J male mice aged 10-14 weeks (Jackson #000664). Under isoflurane anesthesia, with the mice in the supine position, the abdomen was prepped, and an ultrasound probe was used to determine the location of the CSMGC, which is ventral to the abdominal aorta, medial to the upper pole of the left kidney. The region was marked, and the focused ultrasound transducer probe was placed over the abdomen at the marked site. Focused ultrasound stimulation was delivered for 5 mins, as 1 minute of stimulation (1.1 MHZ, 200 mV per pulse, 150 burst cycles, 500 μs burst period) followed by a 30 sec rest period. Immediately following FUS or sham stimulation lipopolysaccharide (0.25 mg/kg) was injected intraperitoneally and 90 mins later mice were euthanized, and blood, spleen, and liver were collected and processed for analysis. FUS stimulation of the CSMGC in C57BL6 mice (n=12) significantly suppressed serum pro-inflammatory cytokine TNF (n=12). FUS stimulation of the CSMGC in C57BL6 mice (n=8 and 10) significantly elevated serum anti-inflammatory cytokine IL-10 compared with sham stimulation. There was also a significant reduction in liver TNF levels in the mice which underwent FUS as compared to sham stimulation. To confirm that FUS effects are due to targeting the CSMGC, we performed FUS vs sham stimulation of the area of the CSMGC in mice with surgically removed ganglionic complex. Gangliectomy was performed in both male and female transgenic mice expressing red fluorescent proteins in the cholinergic nerves—ChAT-tdTomato. In these mice cholinergic innervations of the ganglion complex appear bright red under the fluorescent stereomicroscope and the CSMGC can be reliably visualized and surgically removed. We performed careful dissections to remove the CSMGC in its entirety without damaging the surrounding vasculature. We allowed the mice to recover for 1 week and then subjected the mice to FUS of the CSMGC area for 5 mins (as mentioned above). 5 mins post FUS, LPS (0.25 mg/kg, i.p.) was administered and 90 mins post LPS mice were euthanized, and serum and liver collected for cytokine analysis. Of note, there was no alteration in serum and hepatic TNF levels in FUS of the CSMGC area as compared to the sham stimulation in male and female mice.

### Example 1

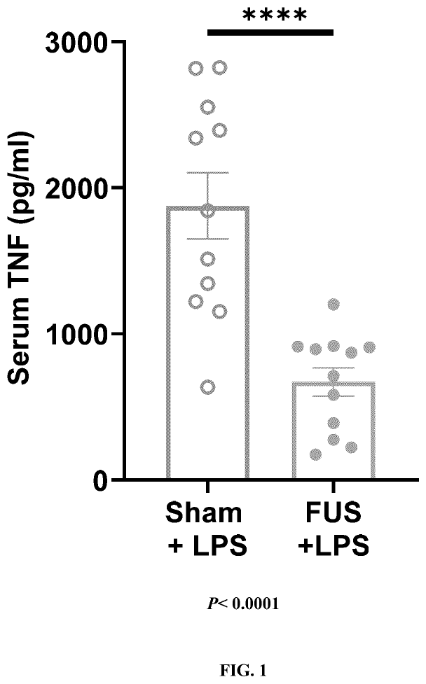

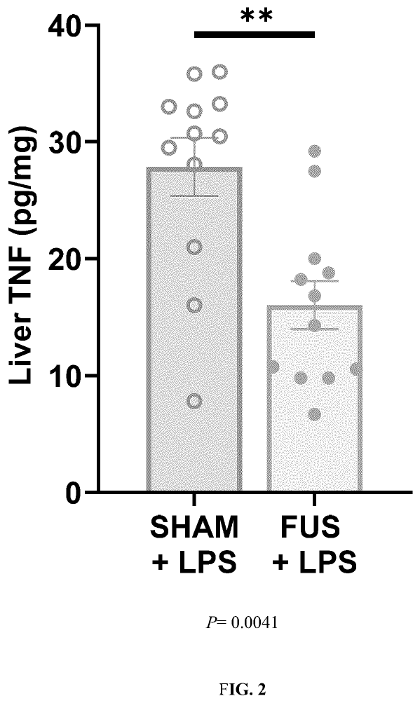

To investigate the hypothesis that stimulation of the neuronal endings and cell bodies within the CSMGC attenuates inflammation, we used commercially available C57BL6/J male mice aged 10-14 weeks (Jackson #000664). Under isoflurane anesthesia, with the mice in supine position, the abdomen was prepped, and an ultrasound probe was used to determine the location of the CSMGC, which is ventral to the abdominal aorta, medial to the upper pole of the left kidney. The region was marked, and the focused ultrasound transducer probe was placed over the abdomen at the marked site. FUS was delivered for 5 mins, as 1 min of stimulation (1.1 MHZ, 200 mV per pulse, 150 burst cycles, 500 μs burst period) followed by a 30 sec rest period. Five mins following FUS or sham stimulation lipopolysaccharide (LPS, Endotoxin, 0.25 mg/kg) was injected intraperitoneally and 90 mins later mice were euthanized, and blood and liver were collected and processed for analysis. FUS stimulation of the CSMGC in C57BL6 mice (n=12) significantly suppressed serum pro-inflammatory cytokine TNF compared with sham stimulation (n=12) (P<0.0001) (FIG. 1). There was also a significant reduction in liver TNF levels in the CSMGC FUS compared with the sham group (P=0.0041) (FIG. 2).

The results are shown in FIG. 1. As can be seen, FUS of the CSMGC attenuated serum (FIG. 1) and hepatic TNF (FIG. 2) levels in murine endotoxemia. This indicates that inflammatory disease states such as, for example, sepsis, rheumatoid arthritis, Crohn's disease, cancer, ankylosing spondylitis, cardiac and cerebral ischemia, vasculitis, Alzheimer's disease, multiple sclerosis, systemic lupus erythematosus, obesity, diabetes, acute liver injury, non-alcoholic steatohepatitis, and other liver diseases, may be treated non-invasively in a mammalian patient (e.g., a human) by applying FUS to the CSMGC of said patient.

### Example 2

To provide further insight, we examined the effect of FUS stimulation applied to the CSMGC or the surgical removal of the CSMGC on the levels of the pro-inflammatory cytokine TNF in mice with endotoxemia. FUS stimulation or sham stimulation was applied in anesthetized male C57BL/6 mice through a probe placed on the abdomen over the area of CSMGC, which was identified using color doppler ultrasound. Pulsed waves at 1.1 MHz and 20 OmV per pulse were delivered for 5 mins. In ganglionectomy experiments, in male ChAT-TdTomato mice, the CSMGC was precisely localized based on endogenous fluorescence associated with cholinergic preganglionic fibers innervating this ganglionic complex. Mice underwent surgical ganglionectomy or sham surgery. Five mins post FUS stimulation, or 7 days post ganglionectomy, mice were injected with lipopolysaccharide (LPS, endotoxin, 0.25 mg/kg, i.p.) and euthanized 90 minutes later, and blood and spleen were collected and processed for cytokine analysis. Compared with sham stimulation, FUS stimulation of the CSMGC (n=8 per group) significantly decreased serum TNF levels (1341±277.7 pg/ml vs 705.6±254 pg/ml, P=0.0006) in endotoxemic mice. There was a significant increase in serum TNF (1360±942.9 pg/ml vs 456.1±274.4 pg/ml, P=0.0281) and in splenic TNF levels (52.41±25.04 pg/mg protein vs ±22.72±8.215 pg/mg protein, P=0.0006) in ChAT-TdTomato mice with a surgically removed CMSGC compared with the sham group (n=8 per group) during endotoxemia. In situ microscopic examination of the CSMGC area in ganglionectomized mice vs controls confirmed the success of the procedure. For the first time these results indicate the anti-inflammatory efficacy of non-invasive FUS stimulation of the CSMGC and reveal the tonic anti-inflammatory function of this ganglionic complex.

In view of these findings, FUS stimulation of a mammalian patient's CSMGC will control the pro-inflammatory cytokine levels and provide a non-invasive method to treat inflammatory disease states in the patient (e.g., a human).

### Example 3

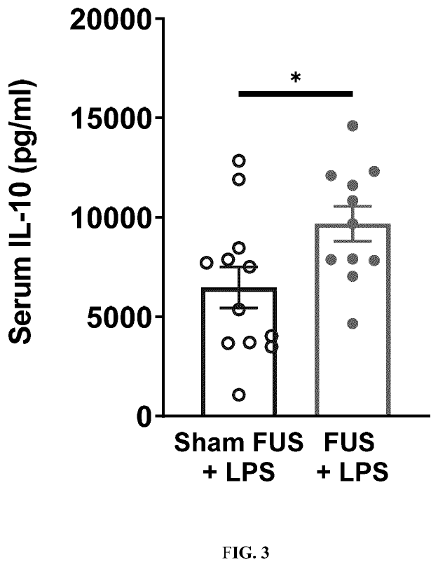

Serum IL-10 is an anti-inflammatory cytokine that is released during inflammation and plays an important role in inflammation regulation. Hence, we examined the effect of FUS of the CSMGC (vs sham stimulation) on serum IL-10 levels in mice during endotoxemia. There was a significant increase of the serum IL-10 levels in mice which received the stimulation as compared to sham stimulated mice (FIG. 3).

### Example 4

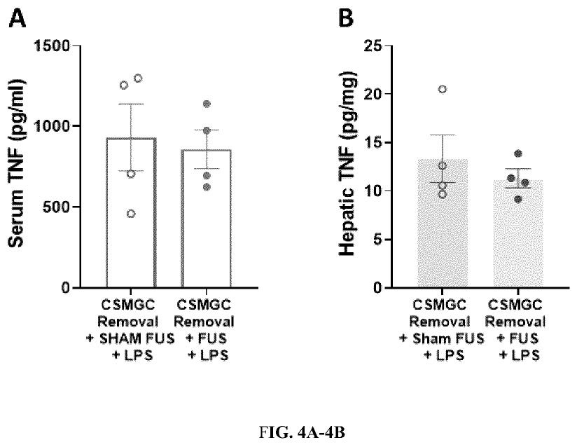

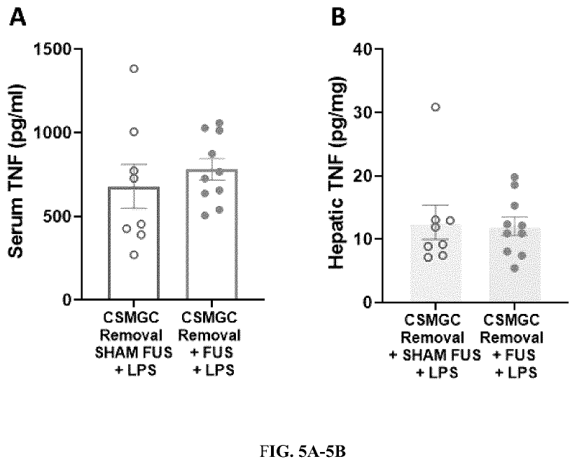

To confirm that FUS effects are due to targeting the CSMGC, we performed FUS vs sham stimulation of the area of the CSMGC in mice with surgically removed ganglionic complex. Gangliectomy was performed in both male and female transgenic mice expressing red fluorescent proteins in the cholinergic nerves—ChAT-tdTomato. In these mice cholinergic innervations of the ganglion complex appear bright red under the fluorescent stereomicroscope and the CSMGC can be reliably visualized and surgically removed. We performed careful dissections to remove the CSMGC in its entirety without damaging the surrounding vasculature. We allowed the mice to recover for 1 week and then subjected the mice to FUS of the CSMGC area for 5 mins (as mentioned above). 5 mins post FUS, LPS (0.25 mg/kg, i.p.) was administered and 90 mins post LPS mice were euthanized, and serum and liver collected for cytokine analysis. Of note, there was no alteration in serum and hepatic TNF levels in FUS of the CSMGC area as compared to the sham stimulation in male (FIG. 4) and female (FIG. 5) mice.

### Discussion

The vagus nerve-based inflammatory reflex is a key physiological regulator of pro-inflammatory cytokines and inflammation. The celiac-superior mesenteric ganglion complex (CSMGC) is an important relay center in the efferent arm of the inflammatory reflex^{4,5}. In the CSMGC, efferent vagus cholinergic fibers and sympathetic preganglionic neurons interact with catecholaminergic neurons, which innervate the spleen and other organs. While significant progress in understanding the role of the efferent vagus nerve and the splenic nerve in the regulation of inflammation has been made, the possibilities to directly and non-invasively target the CSMGC to control inflammation remained unknown.

Our results indicate that the CSMGC is a new, previously unexplored, location that can be used to control inflammation therapeutically. Previously, optogenetic stimulation of cholinergic neuronal bodies in the DMN has been shown to significantly suppress pro-inflammatory cytokine levels in ChAT-ChRh2-eYFP mice during endotoxemia⁴. However, stimulation of cholinergic axonal endings, including those innervating the CSMGC, has not been previously explored in inflammation research. Our results show that FUS stimulation of cholinergic axonal terminals and the adrenergic cell bodies in the CSMGC reliably and significantly reduced serum proinflammatory cytokine levels and increased anti-inflammatory cytokine levels following endotoxemia. It also attenuated cytokines in other organs innervated through the CSMGC, such as the liver. While in the absence of the CSMGC following ganglionectomy, FUS of the CSMGC area did not alter serum and hepatic TNF levels post endotoxemia. Our results also demonstrate that removing the CSMGC results in increased serum and splenic pro-inflammatory cytokine levels in mice with endotoxemia.

The data presented herein indicate that applying FUS non-invasively to the CSMGC can be used to treat and control inflammatory disease states in a mammalian patient or subject (e.g., a human).

While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

### REFERENCES CITED

### Addin En.Reflist

1. Tracey K J. The inflammatory reflex. Nature. 2002;420:853-9. 12490958
2. Pavlov V A and Tracey K J. The vagus nerve and the inflammatory reflex—linking immunity and metabolism. Nature reviews Endocrinology. 2012;8:743-54. 23169440 PMC4082307
3. Kressel A M, Tsaava T, Levine Y A, Chang E H, Addorisio M E, Chang Q, Burbach B J, Carnevale D, Lembo G, Zador A M, Andersson U, Pavlov V A, Chavan S S and Tracey K J. Identification of a brainstem locus that inhibits tumor necrosis factor. Proceedings of the National Academy of Sciences of the United States of America. 2020;117:29803-29810. 33168718
4. Pavlov V A and Tracey K J. Neural circuitry and immunity. Immunologic research. 2015;63:38-57. 26512000 PMC4743890
5. Berthoud H R and Powley T L. Characterization of vagal innervation to the rat celiac, suprarenal and mesenteric ganglia. Journal of the autonomic nervous system. 1993;42:153-69. 8450174
6. Bloom O, Tracey K J and Pavlov V A. Exploring the vagus nerve and the inflammatory reflex for therapeutic benefit in chronic spinal cord injury. Current opinion in neurology. 2022. 35102123
7. Pavlov V A and Tracey K J. Bioelectronic medicine: Preclinical insights and clinical advances. Neuron. 2022;110:3627-3644. 36174571
8. Koopman F A, Chavan S S, Miljko S, Grazio S, Sokolovic S, Schuurman P R, Mehta A D, Levine Y A, Faltys M, Zitnik R, Tracey K J and Tak P P. Vagus nerve stimulation inhibits cytokine production and attenuates disease severity in rheumatoid arthritis. Proceedings of the National Academy of Sciences of the United States of America. 2016;113:8284-9. 27382171 PMC4961187
9. Bonaz B, Sinniger V, Hoffmann D, Clarencon D, Mathieu N, Dantzer C, Vercueil L, Picq C, Trocme C, Faure P, Cracowski J L and Pellissier S. Chronic vagus nerve stimulation in Crohn's disease: a 6-month follow-up pilot study. Neurogastroenterology and motility: the official journal of the European Gastrointestinal Motility Society. 2016;28:948-53. 26920654
10. Zachs D P, Offutt S J, Graham R S, Kim Y, Mueller J, Auger J L, Schuldt N J, Kaiser C R W, Heiller A P, Dutta R, Guo H, Alford J K, Binstadt B A and Lim H H. Noninvasive ultrasound stimulation of the spleen to treat inflammatory arthritis. Nature communications. 2019;10:951.
11. Ahmed U, Graf J F, Daytz A, Yaipen O, Mughrabi I, Jayaprakash N, Cotero V, Morton C, Deutschman C S, Zanos S and Puleo C. Ultrasound Neuromodulation of the Spleen Has Time-Dependent Anti-Inflammatory Effect in a Pneumonia Model. Frontiers in immunology. 2022;13:892086. 35784337 PMC9244783
12. Zanos S, Ntiloudi D, Pellerito J, Ramdeo R, Graf J, Wallace K, Cotero V, Ashe J, Moon J, Addorisio M, Shoudy D, Coleman T R, Brines M, Pulco C, Tracey K J and Chavan S S. Focused ultrasound neuromodulation of the spleen activates an anti-inflammatory response in humans. Brain stimulation. 2023;16:703-711. 37055009 PMC10330863
13. Cotero V, Fan Y, Tsaava T, Kressel A M, Hancu I, Fitzgerald P, Wallace K, Kaanumalle S, Graf J, Rigby W, Kao T J, Roberts J, Bhushan C, Joel S, Coleman T R, Zanos S, Tracey K J, Ashe J, Chavan S S and Pulco C. Noninvasive sub-organ ultrasound stimulation for targeted neuromodulation. Nature communications. 2019;10:952. 30862827
14. ter Haar G and Coussios C. High intensity focused ultrasound: physical principles and devices. International journal of hyperthermia. 2007;23:89-104.
15. Peck M C, Ahmed M, Napoli A, ten Haken B, McWilliams S, Usiskin S I, Pinder S E, van Hemelrijck M and Douck M. Systematic review of high-intensity focused ultrasound ablation in the treatment of breast cancer. Br J Surg. 2015;102:873-82; discussion 882. 26095255
16. Zhou Y F. High intensity focused ultrasound in clinical tumor ablation. World J Clin Oncol. 2011;2:8-27. 21603311 PMC3095464
17. Lyon P C, Rai V, Price N, Shah A, Wu F and Cranston D. Ultrasound-Guided High Intensity Focused Ultrasound Ablation for Symptomatic Uterine Fibroids: Preliminary Clinical Experience. Ultraschall Med. 2020;41:550-556. 31238385
18. Bao S C, Li F, Xiao Y, Niu L and Zheng H. Peripheral focused ultrasound stimulation and its applications: From therapeutics to human-computer interaction. Frontiers in neuroscience. 2023;17:1115946. 37123351 PMC10140332
19. di Biase L, Falato E and Di Lazzaro V. Transcranial Focused Ultrasound (tFUS) and Transcranial Unfocused Ultrasound (tUS) Neuromodulation: From Theoretical Principles to Stimulation Practices. Frontiers in neurology. 2019;10:549. 31244747 PMC6579808
20. Cotero V, Graf J, Miwa H, Hirschstein Z, Qanud K, Huerta T S, Tai N, Ding Y, Jimenez-Cowell K, Tomaio J N, Song W, Devarajan A, Tsaava T, Madhavan R, Wallace K, Loghin E, Morton C, Fan Y, Kao T-J, Akhtar K, Damaraju M, Barenboim L, Maietta T, Ashe J, Tracey K J, Coleman T R, Di Carlo D, Shin D, Zanos S, Chavan S S, Herzog R I and Pulco C. Stimulation of the hepatoportal nerve plexus with focused ultrasound restores glucose homoeostasis in diabetic mice, rats and swine. Nature biomedical engineering. 2022;6:683-705.

We claim:

1. A non-invasive method to treat an inflammatory disease state in a mammalian patient comprising applying noninvasive focused ultrasound stimulation (FUS) to the patient's celiac-superior mesenteric ganglion complex (CSMGC) in an amount effective to treat the inflammatory disease state in a mammalian patient.

2. The method of claim 1, wherein the inflammatory disease state is one or more of sepsis, endotoxemia, metabolic endotoxemia in obesity and type 2 diabetes, septicemia, septic shock, rheumatoid arthritis, Crohn's disease, cancer, ankylosing spondylitis, cardiac ischemia, cerebral ischemia, vasculitis, Alzheimer's disease, multiple sclerosis, systemic lupus erythematosus, obesity, asthma, diabetes, acute liver injury, non-alcoholic steatohepatitis, and liver disease.

3. The method of claim 1, wherein application of FUS to the patient's CSMGC suppresses tumor necrosis factor (TNF) levels and/or elevates interleukin-10 (IL-10) levels in the patient.

4. The method of claim 1, wherein application of FUS to the patient's CSMGC does one or more of the following: suppress tumor necrosis factor (TNF) levels in the patient's serum, suppress TNF levels in the patient's liver, and elevate interleukin-10 (IL-10) levels in the patient's serum.

5. The method of claim 1, wherein the mammalian patient is a human.

6. The method of claim 2, wherein the inflammatory disease state is endotoxemia, metabolic endotoxemia in obesity and type 2 diabetes, sepsis, and acute liver injury.

7. A method of one or both of suppressing tumor necrosis factor (TNF) levels in a mammalian patient and increasing interleukin-10 (IL-10) levels in the mammalian patient comprising applying noninvasive focused ultrasound stimulation (FUS) to the patient's celiac-superior mesenteric ganglion complex (CSMGC) in an amount effective to suppress TNF levels and/or increase IL-10 levels in a mammalian patient.

8. The method of claim 7, wherein TNF levels are suppressed in the mammalian patient's liver and/or serum.

9. The method of claim 7, wherein IL-10 levels are increased in the mammalian patient's serum.

10. The method of claim 7, where in mammalian patient has an inflammatory disease state.

11. The method of claim 10, wherein the inflammatory disease state is one or more of sepsis, endotoxemia, metabolic endotoxemia in obesity and type 2 diabetes, septicemia, septic shock, rheumatoid arthritis, Crohn's disease, cancer, ankylosing spondylitis, cardiac ischemia, cerebral ischemia, vasculitis, Alzheimer's disease, multiple sclerosis, systemic lupus erythematosus, obesity, diabetes, asthma, acute liver injury, non-alcoholic steatohepatitis, and liver disease.

12. The method of claim 7, wherein the mammalian patient is a human.

13. A method of enhancing an inflammatory response in a mammalian patient comprising applying noninvasive focused ultrasound stimulation (FUS) to the patient's celiac-superior mesenteric ganglion complex (CSMGC) in an amount effective to inhibit or ablate neurons and nerve endings in the CSMGC of a mammalian patient.

14. The method of claim 13, wherein application of FUS to the mammalian patient's CSMGC increases serum levels of tumor necrosis factor (TNF) in the mammalian patient and/or decreases serum levels of interleukin-10 (IL-10) in the mammalian patient.

15. The method of claim 13, wherein the mammalian patient is a human.
